(19) United States
(12) Patent Application Publication
(10) Pub. No.: US 2022/0079253 A1
(43) Pub. Date: Mar. 17, 2022

(54) Breast separating and cooling device and method

(71) Applicant: Shunda R. Taylor, Eatonville, FL (US)

(72) Inventor: Shunda R. Taylor, Eatonville, FL (US)

(21) Appl. No.: 17/020,980

(22) Filed: Sep. 15, 2020

(51) Int. Cl. A41C 3/00

(52) U.S. Cl. CPC A41C 3/005

(57) ABSTRACT

A device for separating and spacing breasts to reduce perspiration is provided. The device is designed primarily for use with a female undergarment, but may be used without an undergarment. The device elevates and separates the breasts from each other. Placement of the device under the breast elevates the breast to separate the skin on the underside of the breast from the skin of the user's torso. Airflow under the breasts is greatly enhanced by the device, with additional airflow provided by way of spaced apertures positioned along the device. The device has a hollow interior which may be filled with a cooling gel.

### BACKGROUND OF THE INVENTION

### 1. Field of the Invention

The present invention generally relates to accessories and devices for use with undergarments. More specifically, the invention relates to a device and method for separating and cooling the breasts to reduce sweating.

### 2. Description of the Prior Art

Bras and other undergarments for women are typically designed with comfort and enhanced appearance as an objective. With regard to comfort, it is often desirable that the breasts do not contact each other in a cleavage area, or that if they do contact each other, that such contact is minimized or occurs with only limited force exerted therebetween. Also, it is desirable to reduce or absorb perspiration between and under the breasts. To that end, it is often desirable to have a somewhat absorbent and soft material adjacent inner surfaces of the breasts in a cleavage area to more comfortably support the breasts and potentially absorb any perspiration away from the skin. However, the use of such absorbent material tends to produce as much sweating as it absorbs, and the material itself becomes heavily soiled with extensive use.

Furthermore, often a more desirable appearance in the cleavage area can be created if the breasts avoid contact or minimize contact in a cleavage area. It is also often beneficial to elevate the breasts somewhat for enhanced appearance. Elevating the breasts by separating them from the user's torso has the added benefit of actually reducing sweating by keeping the skin under the breasts cooler.

### SUMMARY OF THE INVENTION

A device for separating and spacing breasts to reduce perspiration is provided. The device is designed primarily for use with a female undergarment, but may be used without an undergarment. The device elevates and separates the breasts from each other. Placement of the device under the breast elevates the breast to separate the skin on the underside of the breast from the skin of the user's torso. Airflow under the breasts is greatly enhanced by the device, with additional airflow provided by way of spaced apertures positioned along the device. The device has a hollow interior which may be filled with a cooling gel.

It is a major object of the invention to provide an improved device for cooling breasts within an undergarment.

It is another object of the invention to provide an improved device for cooling breasts which lifts and separates the breasts from the torso of the wearer.

It is another object of the invention to provide an improved device for cooling breasts which spaces the breast from the torso of the wearer.

It is another object of the invention to provide an improved device for cooling breasts which is hollow and contains a cooling substance.

It is another object of the invention to provide an improved device for cooling breasts which has apertures to promote airflow.

### BRIEF DESCRIPTION OF THE DRAWINGS

FIG. 1 shows a side view of the device positioned under a human breast.

FIG. 2 shows a perspective view of the device.

FIG. 3 shows a perspective view of the device in position including an optional component.

### DETAILED DESCRIPTION OF THE PREFERRED EMBODIMENTS

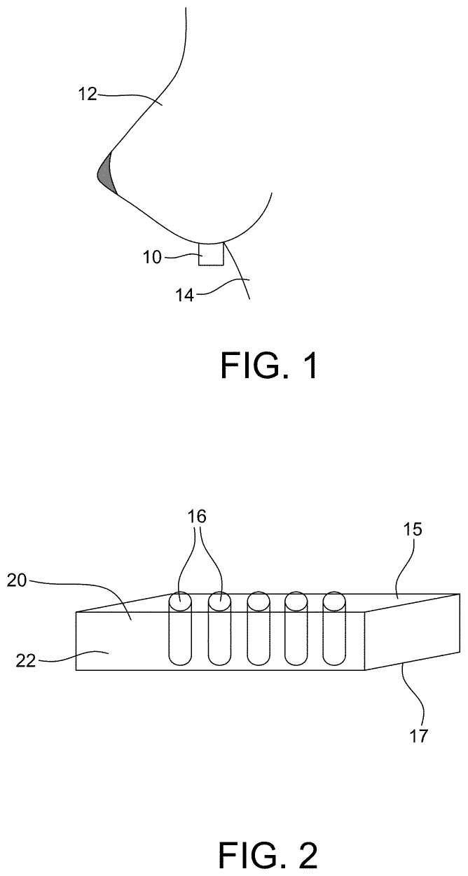

Referring now to FIG. 1, illustrated therein is the device of the invention positioned on a human breast. The device 10 as shown has an elongated substantially rectangular profile but may be optionally contoured for a more comfortable fit. For example, the device 10 may have an arcuate shape, with a cross section being oval as opposed to rectangular. The device 10 is intended to lift the underside of the breast 12 away from the torso 14 of the user in order to allow some air passage therethrough. The amount of lift is determined by the spacing between upper surface 15 and lower surface 17. Thus, the spacing of the breast 12 from the torso provides a cooling effect and allows for the evaporation of any perspiration that does occur. This action also has the benefit of reducing the amount of soil in undergarments. Also, spacing reduces the amount of skin on skin beneath an undergarment, which reduces odor and the buildup of bacteria beneath the breast 12.

Airflow is further enhanced by a series of spaced apertures or bores 16 formed in the device 10, the bores 16 extend across the device 10 and generally perpendicular to the lengthwise direction. As shown, the series of bores 16 are spaced lengthwise but a number of smaller bores extending across the device 10 in any regular pattern may alternatively be used. As can be appreciated, the bores 16 are essentially vent holes that allow air to pass therethrough, resulting in a cooling effect. While some of the apertures 16 will be blocked at one or both ends as some portions of the device 10 will be flush against the user's skin, some air will pass therethrough, this action creating a further cooling effect.

The device 10 may be made from any semi-rigid or slightly pliable durable material, but preferably is made from a hypoallergenic material. Cellulose acetate propionate is ideal for this purpose as it is both hypoallergenic and lightweight. Weight saving is further enhanced by the hollow interior 20 of the device 10. The interior of the device 10 may be filled with refrigerant gel 22 or similar material. When cooled, the gel 22 will provide a cooling effect for at least a few hours. Gels containing ammonium nitrate, calcium ammonium nitrate, or urea may also be used, as these are common ingredients in gel packs.

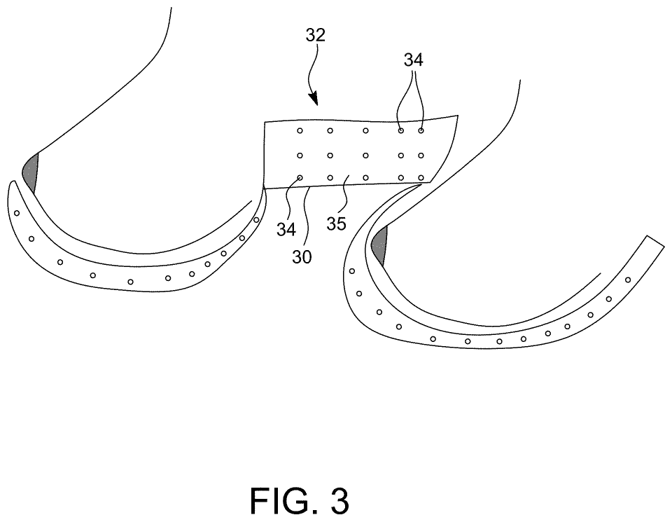

For women with larger breasts, separation of the breasts aids in cooling and evaporation of perspiration. To that end, another component may be added to the device 10 in the form of a separator 30 as can be seen in FIG. 3. Separator 30 has an arcuate rear side 32, with bores or vent apertures 34 extending from the front 36 to the rear 32 of the separator 30. The separator 30 is substantially flat and rectangular and preferably has a concave profile, or is otherwise contoured to more closely conform to the shape of the sternum of the user. The central portion 35 of the separator may be raised to provide additional separation of the breasts. As is the case with the other components of the device 10, the interior of the separator 30 is hollow so that a cooling gel or the like may be contained therein. The separator 30 is sized and shaped for positioning between and under the breasts and by way of its raised profile separates the breasts.

In use, the user may first optionally cool the device 10 in a refrigerator or by other means prior to positioning the device 10. Positioning the device 10 is accomplished by placing the device 10 under the breast 12 and sliding upwards to the area where the breast 12 extends from the torso 14 as shown in FIG. 1. Separator 30 may optionally be used for women with larger breasts or to provide additional lift, spacing, and cooling. The separator 30 is to be positioned on the user's sternum. Regular undergarments may then be worn over the breast. The device 10 may be used with no undergarments and still function to provide a cooling effect.

It is to be understood that the present invention is not limited to the sole embodiment described above, but encompasses any and all embodiments within the scope of the following claims:

I claim:

1. A cooling device for use with a human breast comprising:
a first component having an elongated main body having upper and lower surfaces and a hollow interior;
a series of bores disposed lengthwise along said upper and lower surfaces and extending between said upper and lower surfaces;
a cooling gel contained within said hollow interior;
said device providing lift and spacing of said breast when positioned under said breast.

2. The device of claim 1 wherein said cooling device includes a second component which is shaped for positioning between the breasts.

3. The device of claim 2 wherein said second component is substantially rectangular and has an array of cooling bores formed therein.

4. The device of claim 3 wherein said second component has a hollow interior filed with cooling gel.

5. The device of claim 4 wherein said cooling gel is a refrigerant.

6. The device of claim 1 wherein said cooling gel is refrigerant.

7. The device of claim 1 wherein said first component is made from a hypoallergenic material.

8. The device of claim 2 wherein said second component is made from a hypoallergenic material.
